(19) United States
(12) Patent Application Publication
(10) Pub. No.: US 2012/0107838 A1
(43) Pub. Date: May 3, 2012

(54) Cancer Diagnostics

(75) Inventors: David John Grainger, Cambridge (GB); David Mosedale, King's Lynn (GB)

(73) Assignee: TOTAL SCIENTIFIC LIMITED, Cambridge (GB)

(21) Appl. No.: 13/259,501

(22) PCT Filed: Mar. 24, 2010
(86) PCT No.: PCT/GB2010/000563

§ 371 Date: Jan. 10, 2012

(30) Foreign Application Priority Data
Mar. 24, 2009 (GB) 0905076.6

(51) Int. Cl. G01N 33/574

(52) U.S. Cl. 435/7.23; 436/501; 435/7.1; 435/7.92

(57) ABSTRACT

The invention concerns kits and methods for the diagnosis, prognosis and monitoring of cancer. In one aspect, there is provided a method for identifying whether or not a mammal is suffering from cancer, wherein the method comprises the steps of: (a) measuring a signal due to a non-IgG immunoglobulin binding to a carbohydrate-containing antigen in a sample from the mammal; and (b) comparing the signal measured in step (a) with a signal due to the non-IgG immunoglobulin binding to the carbohydrate-containing antigen in one or more samples from one or more mammals known to have cancer and/or with a signal due to the non-IgG immunoglobulin binding to the carbohydrate-containing antigen in one or more samples from one or more healthy mammals.

This invention relates to kits and methods for the diagnosis, prognosis and monitoring of cancer.

Cancer is the leading cause of death worldwide (7.9 million deaths in 2007; 13% of all deaths worldwide; WHO Factsheet #297 July 2008). Lung cancer is the most prevalent (1.4 million deaths) followed by stomach cancer (866,000 deaths) and colon cancer (677,000 deaths). Despite significant improvements in the standard of care in developed countries, the number of deaths due to cancer globally is expected to double by 2040.

According to the WHO, about one-third of the cancer burden could be eliminated if cases were detected and treated early, before metastasis (invasion of the tumour to distant anatomical sites) can occur. For many cancers, education (to help self-diagnosis of lumps and sores, for example) can make a significant contribution. However, for other cancers, such as colon cancer, symptoms are often not evident until the tumour is well advanced, and the earliest systems are usually non-specific (such as weight loss and fatigue).

As a result, screening programmes have been established for several of the most common cancers, including mammography for breast cancer, cytology (a “pap smear”) for cervical cancer, and colonoscopy for colon cancer. All of these screening approaches are labour intensive and costly to perform, while colonoscopy is additionally unpleasant for the subject and carries a risk of severe complications due to bowel perforation in a small fraction of cases (estimated to be between 1 in 1000 and 1 in 6000 depending on the particular surgeon).

There is, therefore, a considerable opportunity to replace these costly screening frameworks with in vitro diagnostic assays. For example in colon cancer, measuring the level of carcinoembryonic antigen (CEA) in the blood is useful for detecting recurrence of disease following resection but has too many false negatives and false positives to be useful for screening. Similarly, fecal occult blood (FOB) tests have around a 2-5% false positive rate, and is only useful for screening when combined with sigmoidoscopy. As a result, diagnostic colonoscopy is considered the gold standard screening method for colorectal cancer, and every individual over 50 is recommended for screening once every 5 to 10 years in the guidelines issued by the American Society for Clinical Oncology. Similarly, annual colonoscopy following successful treatment is now recommended (since intensive surveillance was associated with a reduction from 37% to 30% in the 5-year mortality rate), but with the exception of serum CEA measurement, no other clinically useful prognostic biomarkers have yet been widely adopted.

Despite a vast literature describing candidate biomarkers associated with solid tumours in a wide variety of tissues, the fact remains that with the exception of prostate specific antigen (PSA) for prostate cancer, thyroglobulin for thyroid tumours and alpha-fetoprotein for hepatocellular carcinoma, no other biomarkers have been described which the necessary sensitivity and specificity for the early detection of solid tumours (Gara et al. (2008) Tunis Med. 86:579-83).

Over the last decade, there has been an explosion in the use of ‘omics’ technologies for biomarker discovery, and with it there has been a rash of manuscripts in the scientific literature describing candidate biomarkers for the detection of tumours in various tissues discovered by methods such as gene expression profiling, proteomics and metabolomics. To date, however, few if any of these new markers have been validated in large scale clinical studies (and the number of plausible candidates is increasing so fast that it is becoming impossible to even attempt to validate the majority of them—as many as 10% of all known proteins have been suggested as candidate markers for cancer somewhere in the literature). The need for clinically useful biomarkers in cancer is, therefore, as pressing as ever.

One promising line of investigation has been the study of cell surface antigens that are differentially expressed by tumour cells compared to their normal progenitors. An important component of the cell surface is the glycochalyx, a corona of oligosaccharide chains presented on cell surface proteins and proteoglycans. There is considerable evidence that the carbohydrate composition of this glycochalyx undergoes subtle changes during cellular transformation from the normal state to the cancerous state.

Changes to protein glycosylation patterns have been found in essentially all tumour cells examined. This aberrant glycosylation often leads to the expression of so-called Tumour-Associated Carbohydrate Antigens (TACAs), which were originally identified through the use of specific monoclonal antibodies. The expression of certain TACAs in some cancers can be as high as 80-100% (e.g. Tn, STn in colorectal carcinoma), much greater than the proportion of the same cancers suffering a deletion or inactivation of oncogenes such as p53 or p16. Such antigens can be useful for the diagnosis and grading of tumours on biopsy samples, but because they are cell-associated antigens they are not detectable in serum and cannot be used for non-invasive detection of solid tumours.

Amongst the most common glycosylation changes are changes in the occurrence of the following carbohydrate structures:

Tn antigen: GalNAc[α1]-Ser/Thr
Sialyl-Tn antigen: Neu5Ac[α2-6]GalNAc[α1]-Ser/Thr
Thomsen-Friedenreich (TF) or T antigen: Gal[β1-3]GalNAc[α1]-Ser/Thr.

Where GalNAc is N-acetyl-galactosamine, Ser is the amino acid serine in a protein chain, Thr is the amino acid threonine in a protein chain, Neu5Ac is N-acetyl-neurominic acid (or sialic acid) and Gal is galactose. Sugar linkages are described in square brackets using conventional carbon numbering to indicate the carbons which are linked through the glycosidic bond, with α and β indicating the stereochemistry of the glycosidic linkage in accordance with common convention.

TF antigen is expressed in about 90% of all human cancers, including colon, breast, bladder, prostate, liver, ovary and stomach, raising the possibility that a “universal cancer marker” may exist, which could be used independently of the type of tumour or affected tissue.

In addition to these three carbohydrate antigens a range of other TACAs have been identified, including sialyl-Lewis X (sLeX; Neu5Ac[α2-3]Gal[β1-4]{Fuc[α1-3]}GlcNAc), sialyl Lewis A (sLeA; Neu5Ac[α2-3]Gal[β1-3]{Fuc[α1-4]}GlcNAc) as well as β1,6 GlcNAc branched products of N acetylglucosaminyltransferase V (GnT-V) and the α-galactosyl epitope (α-gal; Gal[α1-3]Gal[β1-4]GlcNAc), where Fuc represent fucose and GlcNAc is N-acetyl-glucosamine.

The expression of these antigens in tumours (determined by biopsy) has also been correlated with patient survival. For example, patients with breast cancer negative for Tn antigen expression were found to have a significantly better survival rate than those that were positive for Tn expression (Tsuchiya et al. (1999) Breast cancer 6:175-80). Additionally, TF antigen expression has been shown to correlate with invasiveness in bladder cancer (Langlilde et al. (1992) Cancer 69219-27) and be associated with increased risk for liver metastasis in colorectal cancer (Cao et al. (1995) Cancer 76:1700-08).

While TACAs themselves are difficult to detect (primarily because, being cell-associated, a biopsy of the tumour is required), and do not therefore make a plausible target for diagnostic screening (when the purpose is to detect early tumours the location of which are not known), it is well known that antibodies are found directed against TACAs (as, indeed, they are against many other carbohydrate structures) (Bohn (1999) Immunol. Lett. 69:317-20). Anti-carbohydrate antibodies have been described as “natural antibodies” and their presence is ubiquitous, their specificity is often relatively lax (recognizing several or even many loosely related carbohydrate epitopes) and they are often low affinity and hence detectable only at low titres.

It is already well known that human blood contains natural antibodies to many of these TACAs, including TF antigen, Tn antigen and α-gal. The levels of some of these natural antibodies have been shown to be different in patients with cancer than in controls. For example, levels of IgG that bind to TF, Tn and especially α-gal were shown to be reduced in patients with breast cancer, and higher levels of IgG vs. TG antigen were associated with better survival time of stage II breast cancer sufferers in a study by Kurtenkov et al (2005) Exp Oncol 27:136-40. Smorodin et al. (2001, Exp. Oncology 23:109-13) reported reduced levels of IgG vs. TF and Tn antigen in gastric cancer patients and lower IgG vs. TF antigen in colorectal cancer patients. Nevertheless, despite these encouraging early reports, measurement of IgGs against TACAs has not found clinical utility because the extent of any difference between cancer patients and controls is relatively small, the variability between individuals is high and as a result the sensitivity and specificity of such tests are insufficient for use as diagnostic or screening tool.

The present literature, however, is restricted to the measurement of IgG class immunoglobulins (which are by far the most commonly studied class). However, antibodies come in four other classes, distinguished by the heavy chain sequence: IgM (μ heavy chain), IgA (α heavy chain), IgE (ε heavy chain) and IgD (δ heavy chain), in addition to the IgGs (with a γ heavy chain). Additionally, the IgGs are classified into four (in human) sub-classes with distinct, but related γ heavy chains designated IgG1, G2, G3 and G4, and the IgAs are classified into two (in-human) sub-classes with distinct, but related a heavy chains designated IgA1 and A2 Prior studies of antibodies against TACAs have used detection reagents whose specificities are poorly defined, or else ones which have been selected to detect all of the IgG subclasses that may be present.

However, we (Mosedale et al. (2006) J Immunol Methods 309:182-91) and others (Hamadeh et al. (1995) Clin Diagn Lab Immunol 2:125-31) have shown that natural antibodies against carbohydrates exist in classes other than IgG. In relatively large cohorts of healthy subjects antibodies against a range of common carbohydrate antigens were restricted to the G2, A, D and M classes. As a result, it is likely that the majority (if not all) of the IgGs against TACAs detected in the previously published studies were actually IgG2s. Prior to this disclosure, it is entirely unknown whether antibodies against TACAs (or other carbohydrate antigens) of other classes (most likely IgA, IgD and IgM) would be different in cancer compared with controls, or whether any such differences would be clinically useful.

According to a first aspect of the present invention, there is provided a method for identifying whether or not a mammal is suffering from, or at risk from, any form of cancer, wherein the method comprises the following steps:
(a) measuring a signal due to a non-IgG immunoglobulin binding to a carbohydrate-containing antigen in a sample (such as a biological sample) from the mammal; and
(b) comparing the signal measured in step (a) with a signal due to the non-IgG immunoglobulin binding to the carbohydrate-containing antigen in one or more samples (such as biological samples) from one or more mammals known to have cancer and/or with a signal due to the non-IgG immunoglobulin binding to the carbohydrate-containing antigen in one or more samples (such as biological samples) from one or more healthy mammals.

Further aspects and features of the invention are detailed below and in the appended claims.

Here we disclose that naturally occurring non-IgG immunoglobulins against carbohydrate antigens can be used to distinguish samples from subjects with cancer from samples taken from healthy subjects. The measurement of non-IgG immunoglobulins against carbohydrate antigens can therefore be used for the diagnosis, screening, prognosis and monitoring of many different cancer types, and kits for the purpose of making such measurements are consequently claimed. The measurement step of the invention is preferably performed in vitro.

The invention comprises a method for identifying individuals suffering from, or at risk of suffering from, any form of cancer, including (but not limited to) breast cancer, colon cancer, stomach cancer, lung cancer, liver cancer, ovarian cancer, skin cancer, testicular cancer, pancreatic cancer, leukemia, head and neck cancers, tumours of the brain or any other tissue known to be affected by malignant transformation. The said method comprises contacting a suitable sample taken from the individual with one or more carbohydrate-containing antigens in such a manner that antibodies in the sample bind to the carbohydrate antigen(s), followed by detection of non-IgG antibodies bound to the antigen(s). The unique features of the method are the use of carbohydrate-containing antigens and the detection of non-IgG antibodies for the purpose of identifying individuals with, without, or at risk of, cancer.

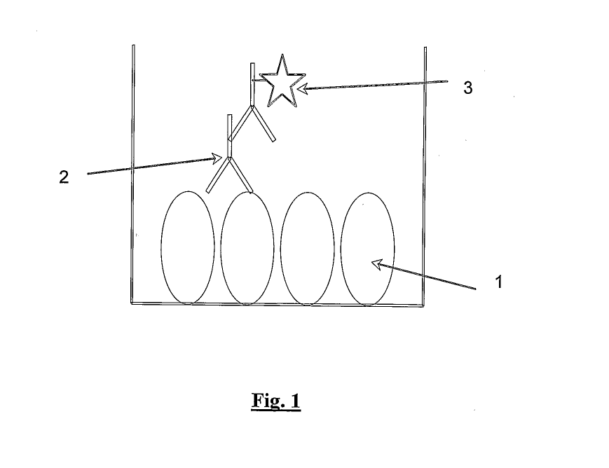

Any technical procedure well known in the art for the purposes of measuring antibody:antigen interactions can be applied for the purpose of implementing the method of the invention in practice. For example, the technique known as enzyme-linked immunosorbant assay (or ELISA) may readily be applied to the implementation of the invention. In this embodiment, the carbohydrate-containing antigen or antigens are coated onto a substrate or surface (typically a commercially-available plastic surface treated in a way to increase the binding of macromolecules), and the sample is applied to the coated substrate. Following a period suitable for binding of any antibodies present in the sample, unbound material is thoroughly washed away. Bound antibodies are detected, typically using anti-antibodies labelled with a suitable enzyme for detection. Anti-antibodies specific for particular classes of human antibodies are well known in the art, and a range of suitable products are commercially available. For the purposes of the present invention, the anti-antibody or anti-antibodies used for detection are selected for their specificity for non-IgG class or classes of human antibodies. The amount of enzyme bound is then quantitated using a suitable substrate, typically a substrate which, on exposure to the enzyme, is converted to a coloured product which can be measure spectrophotometrically. The arrangement of a typical ELISA suitable for practicing the method of the present invention is shown in FIG. 1.

Non-IgG antibodies against a wide range of carbohydrate antigens are different in samples from subjects with cancer compared to healthy subjects. As a result any suitable carbohydrate antigen may be used in accordance with the method of the invention. A suitable carbohydrate antigen is defined as one where the level of non-IgG antibodies binding to that antigen are different in samples from individuals with cancer, or at risk of cancer, compared to those from healthy control individuals. It is important to note that this is not a tautologous definition (it does not say, for example, that the method for defining useful antigens is to test if they are useful) because the key step defining the present invention is the substantial restriction of the search space for useful antigens to those antigens with a carbohydrate component and where the binding antibodies are not IgG class. Antigens with a carbohydrate component compose only a tiny fraction (much less than 0.1%) of all possible antigens, and consequently in providing such a restriction which antigens are likely to be useful, the method makes a useful and inventive contribution.

Preferably, the carbohydrate-containing antigen or antigens are TACAs. Exemplary carbohydrate-containing antigen or antigens according to the invention may be selected from the following table:

| Name | Structure |
|---|---|
| α-gal trisaccharide | Gal[α1-3]Gal[β1-4]GlcNAc- |
| Tn | GalNAc-O-Ser/Thr |
| Sialyl-Tn | NeuAc[α2-6]GalNAc-O-Ser/Thr |
| Lewis-A | Gal[β1-3]{Fuc[α1-4]}GlcNAc- |
| Lewis-X | Gal[β1-4]{Fuc[α1-3]}GlcNAc- |
| Sialyl-Lewis-A | NeuAc[α2-3]Gal[β1-3]{Fuc[α1-3]}GlcNAc- |
| Sialyl-Lewis-X | NeuAc[α2-3]Gal[β1-4]{Fuc[α1-3]}GlcNAc- |
| TF antigen | Gal[β1-3]GlcNAc- |

Other suitable carbohydrate-containing antigen or antigens according to the invention include P1 antigen, Blood group H, Lewis-B, Blood group A trisaccharide, Gal_{α1-2}Gal, Gal_{α1-3}Gal_{β1-3}GlcNAc, Gal_{α1-3}Gal and Gal_{α1-3}Gal_{β1-4}GlcNAc_{β1-3}Gal_{β1-4}Glc.

It will be evident that similar results, useful for the purposes of the present invention, can be obtained using a variety of compounds, analogs and derivatives of the core antigenic oligosaccharides listed here. For example, many of the oligosaccharides can be extended without affecting their antigenic properties, such that the pentasaccharide Gal[α1-3]Gal[β1-4]GlcNAc[β1-3]Gal[β1-4]Glc, which incorporates the α-gal trisaccharide, yields almost indistinguishable results when used according to the method of the invention compared with the shorter α-gal. Similarly, the antigenic oligosaccharide can be compounded with other structural elements, such as a protein or peptide for the purposes of controlling the presentation or physical properties of the carbohydrate-antigen. For example, the oligosaccharides may be conjugated to serum albumin (a protein against which antibodies are not commonly present, and so will not influence the determination of antibodies against an oligosaccharide conjugated to it) to assist in the immobilization of the antigen onto a substrate in an ELISA assay.

It is important to note that the natural antibodies which bind to carbohydrate-containing antibodies often have relatively lax specificity, and consequently a range of structurally related oligosaccharides yield essentially identical results when used to determine the level of non-IgG antibodies present in a wide range of samples, and consequently can readily be substituted for one another when used in accordance with the method of the invention. For the purposes of this specification “essentially identical” means that the signal obtained with the two related oligosaccharides under identical experimental conditions with a panel of samples are correlated with a correlation coefficient of at least 0.8. Such oligosaccharide antigens may be freely substituted with those explicitly disclosed here, in accordance with the method of the invention.

Optionally, the presence of non-IgG antibodies against a multiplicity of carbohydrate-containing antigens may be determined in accordance with the method of the invention. The levels of non-IgG antibodies against several different oligosaccharides in the same sample may be determined serially or in parallel using either conventional one-analyte-at-a-time methods (for example, ELISA assays in multiple wells, each coated with a different antigen and exposed to replicate aliquots of the same sample) or multiplex methods (for example, using dye-encoded beads or barcoded microparticles each coated with a different antigen and then exposed simultaneously to the same sample). The data which is obtained can then be combined using methods well known in the art in order to classify the individual from which the sample was taken as having, or being at risk of, cancer or else as being healthy. For example, the data may be analysed using multivariate modeling methods, including (but not limited to) Principal Component Analysis (PCA), Projection to Latent Structures (PLS), genetic algorithms and similar methods for identifying multivariate diagnostic signatures within large datasets. Alternatively, the data may be queried using rules-based paradigms, to develop clinically useful classifiers.

Alternatively, the presence of non-IgG antibodies against a multiplicity of different carbohydrate-containing antigens may be determined simultaneously in the same assay, where (without any method of determining which antibodies are bound to which antigen) multiple carbohydrate-containing antigens are mixed and coated onto the same substrate. The single output from such an assay is then used to classify the individual from whom the sample was taken as either suffering from, or at risk of, cancer, or else as healthy.

The amount of carbohydrate-containing antigen (in terms of the molar concentration of the carbohydrate antigen in any instance where multiple instances of the antigen motif are present within a single molecule, such as is the case with an albumin protein molecule conjugated with several identical oligosaccharides) is potentially important, and the optimal coating concentration in order to optimize the diagnostic potential of the assay in the required clinical setting must be determined using pilot experiments of the kind well known in the art. The coating density of the antigen is important because natural antibodies are bivalent (or, in the case of IgM, pentavalent) and so are capable of binding more than one molecule of antigen simultaneously provided the antigen coating density is sufficiently high. Thus, depending on the higher the coating density of the antigen, the greater the binding capacity of relatively lower affinity antibodies within the sample. In contrast, lower coating densities will favour the binding of high affinity antibodies (binding through only a single complementarity-determining region (CDR)). Preferably, the coating density will be in the range 5 pmole/cm² to 3.5 nmole/cm².

The absolute amount of antigen coated (in molar terms) relative to the sample volume (irrespective of the surface area of the substrate) is also potentially important, and the optimal coating amount in order to optimize the diagnostic potential of the assay in the required clinical setting must be determined using pilot experiments of the kind well known in the art. The coating amount is important because the amount of antibody capable of binding the antigen which is present in the serum will affect the signal obtained depending on the amount of antigen which is presented in the assay. In particular, where antibodies of different classes directed against the same antigen are present in a sample, there will be a competition between those antibodies for binding. The outcome of this competition will depend on the relative affinities of the various pools of antibodies, but also on their relative amounts. Hence, in circumstances where there is a lot of IgG present (for example), and a smaller amount of IgA, then small amounts of antigen in the assay will favour detection of the IgG, whereas a large amount of antigen in the assay will increase the detection of the less abundant species (IgA in this example). Preferably, the coating amount will be in the range 5 pmoles to 3.5 nmole per 50 μl of sample used.

Prior to exposure of the antigen to the sample, any non-specific binding sites on the substrate should be blocked. Typically, this blocking step is performed by exposing the antigen-coated substrate to high concentrations of macromolecules (such as protein, DNA or carbohydrates) which bind to, and block, the high copy number, low affinity binding sites on the substrate. Typically, the substrate is washed prior to blocking (for example, with three brief washes in phosphate-buffered saline (PBS) containing 0.05% Tween-20) and then exposed to the blocking solution. Examples of suitable blocking solutions would include 0.1-5% bovine serum albumin (BSA) in PBS, preferably 0.5% BSA in PBS, or 1-10% sucrose in PBS containing 1-10% Tween-20, preferably 5% sucrose in PBS containing 5% Tween-20. Typically, the substrate is exposed to the blocking solution for between 15 mins and 4 hours, preferably around 1 hour. Thereafter, the substrate is typically washed to remove the blocking solution prior to exposure to the sample.

The carbohydrate-containing antigen is then exposed to the sample. The sample can be any biological fluid from the individual that contains immunoglobulins, including serum, plasma, whole blood and any other processed derivative of blood (such as a purified immunoglobulin fraction). Samples may also include saliva, tears, mucous, blister fluid and any other secretions, excluding only secretions from a known tumour, or tissue affected by a known tumour. Thus, the invention in one aspect explicitly excludes analysis of the tumour cells (such as from a biopsy) or the products of those cells. Preferably, the sample will be serum. Serum for use according to the present invention may be prepared by any of the methods commonly used for preparing serum (such as the use of serum separator tubes) but the method selected must be consistently applied to all samples analysed according to the method of the invention.

The sample may be diluted prior to exposure to the carbohydrate-containing antigen. The optimal dilution in order to optimize the diagnostic potential of the assay in the required clinical setting must be determined using pilot experiments of the kind well known in the art. In particular, where antibodies of different classes directed against the same antigen are present in a sample, there will be a competition between those antibodies for binding. The outcome of this competition will depend on the relative affinities of the various pools of antibodies, and hence on their absolute concentration in the sample. Hence, in circumstances where there is a lot of low affinity IgG present (for example), and a smaller amount of high affinity IgA, then high dilution of the sample will favour detection of the IgA, whereas a concentrated sample in the assay will increase the detection of the lower affinity species (IgG in this example). Preferably, the any dilution of the sample will be in the range neat (that is, undiluted sample) to a 1:100 dilution of the sample. Note that these represent relatively little dilution compared to what is typically used in ELISA assays in the art, reflecting the relatively low affinity of the natural antibodies against carbohydrate-containing antigens.

Where the sample is diluted, an appropriate diluent must be selected. Any diluent commonly used in the art may be selected, but appropriate experiments well known in the art should be performed to select the diluent that leads to optimum diagnostic potential of the assay in the required clinical setting. Appropriate diluents will preferably be selected from among a group consisting of pooled normal human serum, phosphate-buffered saline (PBS), PBS containing between 0.005% and 1% of a non-ionic detergent such as Tween-20, high purity water, hypertonic PBS containing up to 500 mM additional salt, such as sodium chloride, PBS containing up to 1M urea or PBS with the pH adjusted to between 5.5 and 8.5 units. More preferably, the diluent is PBS.

The sample (diluted if appropriate) is exposed to the carbohydrate-containing antigen under conditions that permit binding of the non-IgG immunoglobulins in the sample to the carbohydrate-containing antigen. Conditions for this incubation, including temperature, time and degree of agitation, should be selected in order to optimize the diagnostic potential of the assay in the required clinical setting must be determined using pilot experiments of the kind well known in the art. In particular, where antibodies of different classes directed against the same antigen are present in a sample, there will be a competition between those antibodies for binding. The outcome of this competition will depend on the kinetics and temperature sensitivity of the binding for each pool. Thus, long incubations will favour interactions with slower kinetics, while shorter incubations will favour more rapid (but possibly thermodynamically less favoured) interactions. Preferably, the sample is incubated with the carbohydrate-containing antigen for between 15 minutes and 4 hours, more preferably for about 2 hours. Preferably, the incubation is performed between 4° C. and 37° C., more preferably around 21° C. Preferably, the incubation is performed with agitation on an orbital shaker (between 0 and 700 rpm), more preferably around 400 rpm.

Following completion of the sample exposure step, unbound material should be efficiently washed away. Conditions for this washing step, including the temperature, number of washes, volume of washes and nature of the washing solution, should be selected in order to optimize the diagnostic potential of the assay in the required clinical setting, and can be determined using pilot experiments of the kind well known in the art. In addition to washing away unbound material, the washing buffer may be selected to increase the stringency of binding (by disrupting weaker, low affinity interactions while leaving in tact stronger, higher affinity interactions). Thus washing buffers containing detergents, or denaturing agents or with hypertonic or hypotonic osmolarity, will affect the relative detection of different pools of antibodies with differing affinities. Appropriate wash solutions will preferably be selected from among a group consisting of phosphate-buffered saline (PBS), PBS containing between 0.01% and 1% of a non-ionic detergent such as Tween-20, high purity water, hypertonic PBS containing up to 500 mM additional salt, such as sodium chloride, PBS containing up to 1M urea or PBS with the pH adjusted to between 5.5 and 8.5 units. More preferably, the wash solution is PBS containing 0.05% Tween-20. Typically, the wash volume is between 4 and 10 times greater than the sample volume used, and the number of washes is between 3 and 5. The higher the concentration of immunoglobulin (whether directed against the selected carbohydrate-containing antigen or not), which will be influenced by the extent to which the sample was diluted, the greater the volume and/or number of washes that will be required. Optionally, a sample known not to contain antibodies specific for the selected carbohydrate-containing antigen can be used to estimate the efficiency of the wash step (since there should be no signal from such a sample unless unbound immunoglobulin was retained through the procedure due to inefficient wash procedures) and hence to select an appropriate wash protocol. Preferably, the washes will be performed between 4° C. and 37° C., more preferably around 21° C. Preferably the duration of each wash will be between 10 seconds and 3 minutes, more preferably around 30 seconds.

Following washing, any bound non-IgG antibody is detected. Detection can be performed using any appropriate reagent, typically an anti-antibody. The selected detection reagent must be specific for one or more class of non-IgG antibody over binding to IgG (or any specific sub-class of IgG), where specificity is defined as at least 100-fold, and more preferably at least 1000-fold, higher affinity for binding to one or more class of non-IgG antibody over binding to IgG. Typically, the anti-antibody will be labelled with an enzyme or other tag (such as a fluorescent dye) which can be quantitated by methods well known in the art. For example, a bound enzyme tag (such as horseradish peroxidase or alkaline phosphatase) can be quantitated by the conversion of a suitable substrate into a coloured product which can itself be quantitated spectrophotometrically.

The conditions for the detection step should be selected so as optimize detection of the non-IgG antibodies from the sample which were captured on the selected carbohydrate-containing antigen. Generally, higher concentrations of detection anti-antibody will yield a higher signal to noise ratio, but care must be exercised to ensure that detection of IgG does not occur (since higher detection antibody concentration will favour lower affinity interactions, such as binding to IgG, over higher affinity interactions, such as binding to the target class of non-IgG immunoglobulins). Typically, the highest concentration of anti-antibody detection reagent that does not result in unintended detection of IgG is preferred.

Typically, the detection reagent is diluted in the wash solution, but other solutions including phosphate-buffered saline (PBS), PBS containing between 0.005% and 1% of a non-ionic detergent such as Tween-20, high purity water, hypertonic PBS containing up to 500 mM additional salt, such as sodium chloride, PBS containing up to 1M urea or PBS with the pH adjusted to between 5.5 and 8.5 units may be used to improve the specificity of the detection reagent for binding to non-IgG immunoglobulins. Preferably the detection reagent is incubated for between 15 mins and 4 hours, more preferably for around 1 hour. Preferably the incubation is performed at between 4° C. and 37° C., more preferably at around 21° C. Preferably, the incubation is performed with agitation on an orbital shaker (between 0 and 700 rpm), more preferably around 400 rpm.

After incubation with the detection reagent, any unbound detection reagent must be washed away. Typically, the same conditions are used for this wash step as for the wash step following exposure of the sample to the carbohydrate-containing antigen. It is important to ensure that essentially all unbound detection reagent is washed away prior to quantitating the amount of bound label.

Alternatively, the specificity of detection of the non-IgG immunoglobulin classes and the subsequent quantitation can be separated into two or more steps. For example, specific mouse monoclonal anti-antibody directed against one or more human non-IgG classes could be used, followed by an anti-mouse detection reagent labelled with an enzyme, radioactivity, fluorescent tag or other quantifiable tag. Such an arrangement may be selected for a number of reasons: due to availability of high quality reagents, to improve the specificity of the detection of only non-IgG immunoglobulins or to increase the signal-to-noise ratio through amplification of the specific signal caused by the multivalent interactions of the two ‘layers’ of antibodies used. It is important, however, when introducing extra ‘layers’ of anti-antibodies into the procedure that the specificity of each and every anti-antibody used is established. For example, the labelled anti-mouse immunoglobulin should not bind to any human immunoglobulins directly, or procedure may (to a greater or lesser degree) inadvertently measure human IgG as well as the non-IgG immunoglobulins.

The bound label is then quantitated by an appropriate method. For example, enzyme-linked detection antibodies are detected by exposing the well to a solution containing a substrate of the enzyme label, which is converted into a product that can readily be detected. Typically, the product is detected spectrophotometrically (for coloured products) or fluorimetrically (for fluorescent products). Alternatively, where the detection reagent was tagged using directly quantifiable label, such as a fluorescent dye, the amount of dye present is quantitated directly, for example using a fluorescent microscope.

It is envisaged that variations on this process can be equally adopted in order to implement the method of the invention, taking into account the same principles. For example, it would be possible to measure the levels of non-IgG immunoglobulin against a particular carbohydrate-containing antigen by using labelled antigen (rather than a labelled detection anti-antibody). In this embodiment, the total immunoglobulin of a non-IgG class is captured onto the substrate (typically using an unlabelled anti-antibody) and then the amount of that antibody pool specific for the particular antigen is determined using the antigen tagged with a label which can readily be quantified (such as an enzyme, radioactivity or a fluorescent dye). This embodiment of the invention may be particularly useful with non-IgG immunoglobulin classes that are present in low absolute amounts in the sample (such as IgE in serum for example).

The selection of appropriate steps and the order in which they are performed in order to effect a measurement of the level of non-IgG immunoglobulin directed against a selected carbohydrate-containing antigen for the purposes of classifying an individual as having, or being at risk of, cancer is not an aspect of the present invention. Any suitable method known in the art may be employed, and different methods may have different advantages for different applications (because of the competition of different antibody pools of different affinities and different immunoglobulin classes, the output from different procedures intended to measure the level of non-IgG immunoglobulin binding to a particular antigen will differ to some degree depending on the method selected). As a result, the precise method to be used is optimized for a particular application by experimentation, adopting approaches well known in the art.

Without compromising the generality of present invention, a preferred embodiment of the invention is an ELISA assay to measure non-IgG immunoglobulin binding to a carbohydrate-containing antigen, in which the antigen is immobilized onto a suitable substrate (which is subsequently blocked for non-specific binding) and then exposed to the sample. After washing away unbound material, the bound non-IgG immunoglobulin is detected using an appropriate detection reagent such as a specific anti-antibody labelled with an enzyme. The amount of label bound is then quantitated, for example by exposing the enzyme to a suitable substrate, and measuring the amount of a coloured product by spectrophotometry.

More preferably, this protocol is performed using a TACA as the carbohydrate-containing antigen. More preferably, the non-IgG immunoglobulin that is detected is IgA.

A preferred embodiment of the invention for classifying subjects as having, or being at risk of, breast cancer is a protocol consisting of the following steps:

1. Coat wells of a microtitre plate with 50-100 pmoles of α-gal linked to human serum albumin in 50 μl of 50 mM Na₂CO₃, pH 9.6.
2. Wash wells three times with PBS containing 0.05% Tween-20.
3. Block wells with PBS containing 5% sucrose and 5% Tween20.
4. Wash wells three times with PBS containing 0.05% Tween-20 and once with PBS.
5. Incubate wells with 50 μl of samples, either neat or diluted up to 100-fold with PBS.
6. Wash wells five times with PBS containing 0.05% Tween-20.
7. Incubate wells with 200 μl of mouse anti-non-IgG-immunoglobulin antibody (such as anti-IgA-immunoglobulin antibody).
8. Wash wells three times with PBS containing 0.05% Tween-20.
9. Incubate wells with 200 μl of horseradish-peroxidase-labelled anti-mouse IgG antibody.
10. Wash wells three times with PBS containing 0.05% Tween-20.
11. Incubate wells with colour substrate.

A preferred embodiment of the invention for classifying subjects as having, or being at risk of, colon cancer is a protocol consisting of the following steps:

1. Coat wells of a microtitre plate with 50-100 pmoles of P1 antigen and/or Lewis-A antigen linked to bovine or human serum albumin in 50 μl of 50 mM Na₂CO₃, pH 9.6.
2. Wash wells three times with PBS containing 0.05% Tween-20.
3. Block wells with PBS containing 0.05% Tween-20 and 0.5% bovine serum albumin.
4. Wash wells three times with PBS containing 0.05% Tween-20.
5. Incubate wells with 50 μl of samples, either neat or diluted up to 100-fold with PBS.
6. Wash wells five times with PBS containing 0.05% Tween-20.
7. Incubate wells with 200 μl of mouse anti-non-IgG-immunoglobulin antibody (such as anti-IgA-immunoglobulin antibody for Lewis-A antigen or anti-IgM-immunoglobulin antibody for P1 antigen), diluted in PBS containing 0.05% Tween-20 and 0.5% bovine serum albumin.
8. Wash wells three times with PBS containing 0.05% Tween-20.
9. Incubate wells with 200 μl of horseradish-peroxidase-labelled anti-mouse IgG antibody, diluted in PBS containing 0.05% Tween-20 and 0.5% bovine serum albumin.
10. Wash wells three times with PBS containing 0.05% Tween-20.
11. Incubate wells with colour substrate.

Optionally, for each sample to be analyzed by the method of the invention, a replicate assay is performed which is identical in all respects with the test assay intended to measure binding of non-IgG antibodies to the carbohydrate-containing antigen, except that the substrate is not coated with any antigen (or is coated with only the carrier portion of the antigen lacking the carbohydrate epitope). The signal from this replicate well (the ‘no coat control’) may then be subtracted from the signal in the test assay to remove that portion of the signal due to non-specific binding of antibodies in the sample to the substrate, carrier, blocking components or any other part of the assay other than the intended carbohydrate antigen. Preferably, such a no coat control assay is performed when the method of the invention is implemented using ELISA methodology.

In the final step, the data which has been obtained is used to classify individuals as either having, or being at risk of, cancer or else healthy. In its simplest form, the data for a single non-IgG immunoglobulin class binding to a single carbohydrate-containing antigen is compared to a threshold, and individuals on one side of the threshold (for example, below the threshold) are classified as having, or being at risk of, cancer while the remaining individuals are classified as healthy. In a more complex scenario, multiple thresholds are applied to the data for a single non-IgG immunoglobulin class binding to a single carbohydrate-containing antigen in order to define levels of risk. For example, individuals with values below the 10^{th} centile are consider at very high risk of having cancer, while those above the 90^{th} centile are consider very likely to be healthy. The remaining individuals lying between the 10^{th} and 90^{th} centiles are not classified by this test.

Alternatively, data from several assays (either performed simultaneously, whether by conventional methods in parallel wells or by utilizing a multiplexing method, or else performed sequentially but on replicate aliquots of the same sample) are used to construct a multivariate ‘signature’ describing the population of non-IgG immunoglobulins in the sample capable of binding to several different carbohydrate-containing antigens. This signature can then be compared with signatures from individuals with cancer and from healthy individuals in order to classify the subject from which the sample was taken according to their risk of having cancer.

It will be evident that the test can provide clinically useful information about risk of having cancer even when the test is unable to provide a perfect classification of the samples. For such an application, the test is considered to have diagnostic power if (when applied to a cohort of samples whose cancer status is known) the number of positive and negative predictions made are greater than would have been achieved by chance on 19/20 occasions (in other words, the p value comparing the distribution of predicted status against actual status in a contingency table, using Fisher's Exact Test is below 0.05). Provided that none of the samples in such a cohort had previously been used during the selection of the antigens to be tested, nor during the optimization of the method to be used, then such a test is an independent validation of the power of the test to classify unknown samples taken from the same underlying population.

A test according to the method of the invention may be used in a number of different ways. For example, the test could be applied as a screen for identifying individuals who have, or who are at risk from, certain forms of cancer for the purposes of early detection among otherwise healthy individuals. In this application, the test is applied to samples taken from the individuals to be screened, and those for whom a positive result is obtained are investigated and monitored for the presence of cancer. Alternatively, a test according to the method of the invention may be used to assist in the diagnosis of malignancy. Early in the development of a solid tumour, the tumour itself may be too small to be detected physically (for example, by palpation) and the symptoms of the disease may be relatively non-specific (such as lethargy, tiredness and weight-loss). In such circumstances, the test is applied to samples taken from an individual presenting with such symptoms, and those for whom a positive result is obtained are investigated further and the result of the test may be used to arrive at a diagnosis of malignancy. Following such a diagnosis, the subject may be treated for the presence of cancer and the availability of such a novel diagnostic test will likely improve the prognosis for the patient by allowing treatment to begin at the earliest possible juncture, potentially even at a time when conventional diagnostics could not have established the presence of the disease.

In yet another alternative, a test according to the method of the invention may be used to predict future risk of cancer. In such circumstances, the test is applied to samples taken from individuals to be assessed, and those for whom a positive test is obtained are considered at higher risk of developing cancer than those for whom a negative result is obtained. Those at higher risk may be monitored more closely, or undergo lifestyle changes intended to reduce the risk of malignancy developing at some later time.

In yet another alternative, a test according to the method of the invention may be used to monitor the response of an individual to a therapy designed to treat or prevent cancers. In such circumstances, the test is applied to individuals undergoing treatment before and after the treatment is initiated. The test may then be applied once or on multiple occasions after treatment has begun, and after the treatment has been completed. In each case, the test is applied to different samples, prepared by the same method, taken from the same individual but at different times. Any change in the result of the test (either qualitatively, compared to some threshold or multivariate signature, or quantitatively in terms of the signal output by the test) is then interpreted in terms of a change in the severity of the current disease status of the individual, or in the risk of developing the disease, or in the risk of recurrence of the disease. This information may then be used to guide the clinical treatment of the subject, to modify the lifestyle of the subject, or to assist in clinical trials of new agents designed to treat or prevent cancers.

All such applications may include determination of the risk of metastasis (that is, the spread of the cancer from its original site to distant tissues, establishing secondary tumours—a behaviour most often associated with poorer prognosis for the patient and the need for more aggressive therapeutic interventions).

Since differential expression of TACAs is known to occur in essentially every tumour type, the applications of the method of the invention are not restricted to any particular type of cancer, but represent a system for screening, diagnosing and monitoring essentially every type of cancer (although not every combination of non-IgG immunoglobulin class and carbohydrate-containing antigen will be useful for every type of cancer, and certain particular pairings of non-IgG immunoglobulin class and carbohydrate-containing antigen may be particularly useful for only a single, or a number of closely related, cancer types).

Without prejudice for the generality of the foregoing, classification of risk for the following cancers are explicitly within the scope of the present application:
Leukemias (including acute lymphoblastic leukemia, acute myeloid leukemia, chronic lymphocytic leukemia, chronic myelogenous leukemia, cutaneous T cell lymphoma, hairy cell leukemia, Hodgkin's lymphoma, Burkitt's lymphoma, non-Hodgkin's lymphoma, Waldenström's macroglobulinemia, multiple myeloma, myelodysplastic syndromes and Sézary's Syndrome);
Cancers of the endocrine system (including adrenocortical cancer, islet cell carcinoma, childhood multiple endocrine neoplasia syndrome, pancreatic cancer, parathyroid tumours, pheochromocytoma and thyroid cancer);
AIDS-related cancers (including AIDS-related lymphoma and Kaposi's sarcoma)
Cancers of the gastrointestinal tract (including anal cancer, colon cancer, cancer of the appendix, eosophogeal cancer, gallbladder cancer, gastric or stomach cancer, gastrointestinal carcinoid tumour, hypopharyngeal cancer, laryngeal cancer, oral cancer (including lip and oral cavity tumours), oropharangeal tumours, pharangeal tumours, rectal cancer, salivary gland cancer, cancer of the small intestine and throat cancer);
Cancers of the central nervous system (including astrocytomas, brain stem gliomas, brain tumours, malignant glioma, ependymoma, medulloblastoma, supratentorial primitive neuroectodermal tumors, hypothalamic gliomas, neuroblastomas, pineal astrocytoma and pineal germinoma);
Breast cancer and carcinomas (including basal cell carcinoma, renal cell carcinoma and other kidney cancers, Merkel cell carcinoma, Wilm's tumour, transitional cell cancer, thymic carcinoma and thymomas);
Cancers of the urogenital tract (including bladder cancer, cervical cancer, endometrial cancer, extragonal germ cell tumours, ovarian cancer, ovarian epitheloid tumours, ovarian germ cell tumours, penile cancer, prostate cancer, uterine sarcoma, testicular cancer, teratoma, gestational trophoblastic tumours (including hydatiform mole), urethral cancer, vaginal cancer and vulvar cancer);
Adenomas (including carcinoid tumours and bronchial adenomas in childhood);
Bone cancer (including osteosarcoma and malignant fibrous histiocytoma);
Sarcoma (including Ewing's sarcoma, Kaposi's sarcoma and rhabdomyosarcoma);
Eye cancer (including retinoblastoma and intraocular melanoma);
Lung cancer (including mesothelioma and malignant mesothelioma, nasal cancer and paranasal cavity tumours, non-small cell lung cancer, pleuropulmonary blastoma and small cell lung cancer);
Liver cancers (including extrahepatic bile duct cancer and hepatocellular carcinoma);
Head and neck cancers;
Cancers of infectious origin (including mycosis fungoides, human papiloma virus-induced tumours, and other virally-induced tumours); and
Skin cancer (including melanoma, skin carcinoma and Merkel cell carcinoma)

Also provided here is a kit for the purpose of diagnosing, predicting the risk of, or monitoring cancer by measuring non-IgG antibodies against carbohydrate-containing antigens in biological samples, such as human serum. Such a kit comprises one or more carbohydrate-containing antigens according to the method of the present invention, immobilised on a suitable substrate such as a microtitre plate well, together with a detection reagent capable of detecting non-IgG antibodies bound to the plate well. Optionally, the kit may also contain additional reagents, such as wash solutions, solutions for the dilution of samples, enzyme substrates and ancilliary reagents common to ELISA kits.

A kit according to the present invention may include the reagents required to measure the levels of non-IgG antibodies binding to more than one carbohydrate-containing antigen. Multiple antigens may be provided coated as a mixture on a single substrate (such as a well of microtitre plate) or else separately on multiple substrates (such as multiple wells of the microtitre plate). Alternatively, the multiple antigens may be provided on a coded substrate, such as those typically used in a multiplexing system (such as dye-encoded beads, barcoded microparticles or spots on an array).

Optionally, a kit according to the present invention may include multiple detection reagents required to measure separately the levels of more than one class of non-IgG antibodies binding to carbohydrate-containing antigens. The detection reagents may all bear the same or similar tags for the purposes of quantitation (such as the enzyme horseradish peroxidase), intended to be used on multiple replicate wells each coated with the same carbohydrate-containing antigen and exposed to replicate aliquots of the same sample, or alternatively the detection reagents specific for different classes of non-IgG antibodies may each bear a distinct and separately quantifiable label (such as fluorescent dyes with unique spectral properties). The possibility of using multiplexed antigens on coded substrates simultaneously with multiplexed detection reagents with coded tags to create a 3-dimensional profile is also envisaged, and consequently claimed.

A preferred embodiment of such a kit comprises wells of a microtitre plate coated with one or more carbohydrate-containing antigens selected from the group consisting of α-gal, Lewis-X, Lewis-A, sialyl-Lewis X, sialyl Lewis A, Tn, Sialyl Tn, TF antigen, P1 antigen, Blood group H, Lewis-B, Blood group A trisaccharide, Gal_{α1-2}Gal, Gal_{α1-3}Gal_{β1-3}GlcNAc, Gal_{α1-3}Gal, and Gal_{α1-3}Gal_{β1-4}GlcNAc_{β1-3}Gal_{β1-4}Glc. The detection reagent may be specific for IgA1, IgA2, total IgA, IgD, IgE or IgM.

For example, the kit may comprise the carbohydrate-containing antigens selected from the group consisting of α-gal, Lewis-A, Sialyl-Lewis-A, Lewis-X, Sialyl-Lewis-X, Tn, Sialyl-Tn and TF antigen. In particular, in the kit the carbohydrate containing antigen may be α-gal, the detection reagent may be specific for total IgA (or IgA1 or IgA2), and the cancer may be breast cancer.

In another example, the kit may comprise the carbohydrate-containing antigens selected from a group consisting of P1 antigen, Lewis-X, Blood group H, Lewis-B, Blood group A trisaccharide and Gal_{α1-2}Gal. In particular, in the kit the carbohydrate containing antigen may be Lewis-A, the detection reagent may be specific for total IgA (or IgA1 or IgA2), and the cancer may be colon cancer.

In a further example, the kit may comprise the carbohydrate-containing antigens from the group consisting of P1 antigen, Blood group A trisaccharide, Gal_{α1-2}Gal, Gal_{α1-3}Gal_{β1-3}GlcNAc, Gal_{α1-3}Gal, and Gal_{α1-3}Gal_{β1-4}GlcNAc_{β1-3}Gal_{β1-4}Glc. In particular, in the kit the carbohydrate containing antigen may be P1 antigen, the detection reagent may be specific for IgM, and the cancer may be colon cancer.

### DEFINITIONS

The term “non-IgG immunoglobulin” refers to any immunoglobulin other than IgG. IgG immunoglobulins are defined by the presence of a y heavy chain (including any of the four variants γ1, γ2, γ3 and γ4, yielding IgG1, IgG2, IgG3 and IgG4 respectively). Any immunoglobulin lacking a g chain is therefore a non-IgG immunoglobulin. IgA (defined by the presence of an α heavy chain), IgD (defined by the presence of a δ heavy chain), IgM (defined by the presence of a μ heavy chain) and IgE (defined by the presence of an ε heavy chain) are explicitly included in the definition of non-IgG immunoglobulin.

The term “carbohydrate” is used to refer to a sugar or sugar derivative, usually consisting of a five, six, seven or eight membered ring composed primarily of carbon with a single oxygen atom in the ring, with one or more hydroxyl substituents on the ring. Typically, the simple sugars have a chemical formula CₙH₂ₙOₙ. However, such sugars may then be modified, through substitution of amino groups for hydroxyl groups (such as in glucosamine compared to glucose), and through methylation, acetylation, sulfation and other similar derivatisation reactions, and all such modified sugars are included with the definition of “carbohydrate” according to the present invention. Specifically, all the sugar residues commonly used in protein glycosylation are explicitly included in the present definition, including galactose, galactosamine, N-acetyl-galactosamine, glucose, glucosamine, N-acetyl-glucosamine, sialic acid, neramininc acid, N-acetyl-neuraminic acid, mannose, fucose, fucosamine, N-acetylfucosamine and xylose. The term carbohydrate as used herein explicitly includes compound combinations of sugar moieties to form oligosaccharides (through glycosidic bonds).

The term “carbohydrate-containing antigen” is used to refer to any compound which comprises one or more carbohydrate moieties, optionally together with other non-carbohydrate moieties, where the only portion recognized to any significant degree by antibodies present in the majority of biological samples is that portion composed of the carbohydrate moieties.

The terms “about”, “around” or “approximately” refer to an interval around the considered value. As used in this patent application, “about X” means an interval from X minus 10% of X to X plus 10% of X, and preferably an interval from X minus 5% of X to X plus 5% of X.

The use of a numerical range in this description is intended unambiguously to include within the scope of the invention all individual integers within the range and all the combinations of upper and lower limit numbers within the broadest scope of the given range.

As used herein, the term “comprising” is to be read as meaning both comprising and consisting of. Consequently, where the invention relates to an item in a kit, this terminology is intended to cover both items in which other components in additions to the ones specified are present and also items that consist only of the components defined.

The following abbreviations, where used, are intended to refer to the following commonly found sugar moieties, whether isolated or as part of an oligosaccharide or other compound according to the context:

| ABBREVIATION | MEANING |
|---|---|
| Gal | Galactose |
| GalNAc | N-acetyl-galactose |
| Glc | Glucose |
| GlcNAc | N-acetyl-glucosamine |
| Neu5NAc | N-acetyl-neuramininic acid; |
| | Sialic acid |
| Fuc | Fucose |

Unless otherwise defined, all the technical and scientific terms used here have the same meaning as that usually understood by an ordinary specialist in the field to which this invention belongs.

Particular non-limiting examples of the present invention will now be described with reference to the following drawing, in which:

FIG. 1 shows the key steps of the method of the invention, implemented as an ELISA.

In the embodiment shown in FIG. 1, carbohydrate-containing antigen or antigens (1) are coated onto a substrate or surface, and the sample is applied to the coated substrate. Human non-IgG immunoglobulin (2) are allowed to bind to the carbohydrate-containing antigen or antigens, and bound human non-IgG immunoglobulin are then detected by an enzyme-labelled anti-human non-IgG immunoglobulin (3). The ELISA method illustrated in FIG. 1 is discussed in further detail above.

### EXAMPLE 1

### Detection of Breast Cancer by the Level of IgA Antibodies Against α-Gal

The levels of IgA antibodies against a range of carbohydrate-containing antigens were determined in a panel of serum samples from individuals with breast cancer and compared with serum samples from healthy controls. For comparison, the levels of IgG against the same antigens were determined.

Methods: Microtite plates (Nunc Maxisorp™) were coated with a range of carbohydrate-containing antigens at 75-250 pmoles/cm² (75-250 pmoles per well). The antigens used were: lewis A, sialyl lewis X, blood group A antigen, blood group B antigen, α-gal and TF antigen (all purchased from Dextra Laboratories and conjugated to either BSA or HSA). Antigens were dissolved in 50 mM sodium carbonate buffer pH 9.6 at 200 nM (protein component) and 50 μl per well was added. Antigen was left to bind for 18 hours at 21° C. Two further series were coated with the carrier portion of the antigens used (‘BSA’ and ‘HSA’ wells).

After coating, unbound antigen was washed away (3× washes of approximately 30 second duration in PBS+0.05% Tween-20; 375 μl per well), and the substrate was blocked with 5% sucrose, 5% Tween-20 in PBS for 1 hour at room temperature with agitation (˜400 rpm on an orbital shaker; 350 μl per well).

After blocking the wells were washed three times with PBS containing 0.05% Tween-20 as previously and then once with PBS alone, and then exposed to the samples (50 μl per well) for 2 hours at room temperature with agitation (˜400 rpm on an orbital shaker). Samples from individuals with cancer (stage II non-metastatic carcinoma of the breast) were compared with samples from individuals without known cancer and otherwise thought to be healthy. Serum was prepared using Becton-Dickinson serum preparation vacutainers. Serum samples were stored at frozen from preparation until assay, without additional freeze-thaw cycles. Samples were diluted 1:100 with PBS immediately prior to loading onto the plate.

After the sample incubation, the wells were washed as previously (except that 5 washes were performed), and then incubated with the first detection reagent. Replicate assays were performed using mouse anti-human IgA (M26013; clone 2D7 from Skybio Ltd, Wyboston, UK) according to the method of the invention, and separately using anti-human IgG2 (M10015; clone GOM1 from Skybio) for comparison. The detection reagents were diluted 1:10,000 in PBS+0.05% Tween-20 and 200 μl per well were dispensed. The plates were incubated with the first detection reagent for 1 hour at room temperature with agitation (˜400 rpm on an orbital shaker).

After the first detection reagent, the wells were washed three times as previously, and then incubated with the second detection reagent. The second detection reagent, horseradish peroxidase labelled goat anti-mouse IgG was diluted 1:10,000 in PBS+0.05% Tween-20 and 200 μl per well was dispensed. The plates were incubated with the second detection reagent for 1 hour at room temperature with agitation (˜400 rpm on an orbital shaker).

After incubation with the detection reagent, the wells were washed three times as previously. The amount of bound label was then quantified by addition of an appropriate substrate (K-Blue™; Skybio; 200 μl per well). After 5 minutes, the reaction was stopped by the addition of 50 μl of 3M HCl. The quantity of coloured product was determined by reading the absorbance of each well at 450 nm. Data from the ‘HSA’ and ‘BSA’ wells were not subtracted, but are presented separately.

Results: Detectable levels of non-IgG immunoglobulins binding to each of the carbohydrate-containing epitopes were found in the majority of samples. The levels of IgA antibodies against α-gal, TF antigen and sialyl lewis X were substantially and significantly lower among the individuals with cancer than among the healthy control individuals.

The levels of IgA antibodies against the other carbohydrate-containing epitopes tended to be lower among the individuals with cancer, but any differences did not reach statistical significance in this experiment.

By comparison, the level of IgG antibodies against the same antigens, including α-gal, were not significantly different between the subjects with cancer and the healthy control individuals.

Conclusions: We conclude that the measurement of non-IgG immunoglobulins binding to carbohydrate-containing epitopes is useful for the detection of cancer. In particular, the detection of IgA antibodies binding to the α-gal epitope, to the TF antigen and to sialyl lewis X epitope are useful for the classification of individuals for the presence of breast cancer.

By contrast, even though the carbohydrate-containing epitopes themselves are known in the prior art to be differentially expressed on cancer cells, including breast cancer cells, measuring IgG against these epitopes (as has been previously suggested; Kurtenkov et al (2005) Exp Oncol 27:136-40) does not yield the diagnostic utility of the present invention.

### EXAMPLE 2

### Detection of Colon Cancer Using Levels of IgA, IgG2 and IgM Antibodies

In this example, we show that colon cancer can be detected using not only IgG2 antibodies (for comparison) but also IgA and IgM antibodies against various different carbohydrate-containing antigens.

Methods: UltraPlex™ two-digit microparticles were used as the substrate for the assay in order to assay the anti-carbohydrate antibodies in multiplex. The microparticles, pre-prepared using bis-1,2-(triethoxysilyl)ethane (BTSE), were coated with a range of carbohydrate-containing antigens at a concentration of 40 μg/ml in phosphate-buffered saline overnight on a rotator at 37° C. After coating, unbound antigen was washed away (3× washes of approximately 1 minute duration in PBS+0.05% Tween-20 containing 0.1% sodium azide; wash buffer), and the microparticles were blocked with wash buffer containing 0.5% bovine serum albumin (blocking buffer) for 1 hour at room temperature on a tube rotator. Blocked microparticles were stored until use at 4° C. Sixteen coating and blocking procedures were carried out using different coded microparticles, one code for each carbohydrate antigen. The coating materials for the sixteen different microparticle codes were:
1) None/blank
2) BSA
3) HSA
4) P1 antigen (B1010)
5) Lewis X antigen (NGP0501)
6) Lewis A antigen (NGP0502)
7) Blood group H (NGP0503)
8) Lewis B antigen (NGP0601)
9) N-Acetyllactosamine (NGP1201)
10) Blood group A trisaccharide (NGP1305)
11) Blood group B trisaccharide (NGP1323)
12) Gal_{α1-2}Gal (NGP2202)
13) Gal_{α1-3}Gal_{β1-3}GlcNAc (NGP2333)
14) Gal_{α1-3}Gal (NGP3203)
15) α-Gal linear B trisaccharide (NGP3334)
16) Gal_{α1-3}Gal_{β1-4}GlcNAc_{β1-3}Gal_{β1-4}Glc (Penta-gal).

These sixteen differently coated microparticles were mixed and loaded into wells of a 96-well filter plate. The microparticle mixture was then washed three times with wash buffer, and then exposed to the samples (50 μl per well) for 2 hours at room temperature with agitation (˜900 rpm on an orbital shaker).

Samples from individuals with colon cancer (stage II or III non-metastatic colorectal cancer) were compared with two groups of control samples from individuals without known cancer and otherwise thought to be healthy. Control group 1 was used to represent a wider demographic, while Control group 2 was used because the serum samples were prepared by exactly the same protocol as the samples from the cancer patients. Serum samples were stored at frozen from preparation until assay, without additional freeze-thaw cycles. Samples were assayed without dilution.

After the sample incubation, the wells containing microparticles were washed as previously (except that 5 washes were performed), and then incubated with the first detection reagent. Replicate assays were performed using mouse anti-human IgA (M26013; clone 2D7 from Skybio Ltd, Wyboston, UK) or mouse anti-human IgM (M02013; clone AF6 from Skybio Ltd, Wyboston, UK) according to the method of the invention, and separately using anti-human IgG2 (M10015; clone GOM1 from Skybio) for comparison. The detection reagents were diluted to 1.33 μg/ml (for anti-human IgA and anti-human IgG2) or to 0.4 μg/ml (for anti-human IgM) in blocking buffer and 100 μl per well were dispensed. The plates were incubated with the first detection reagent for 1 hour at room temperature with agitation (˜900 rpm on an orbital shaker).

After the first detection reagent, the wells were washed three times as previously, and then incubated with the second detection reagent. The second detection reagent, alexafluor 594 labelled goat anti-mouse IgG, was diluted to 5 μg/ml in blocking buffer and 100 μl per well was dispensed. The plates were incubated with the second detection reagent for 1 hour at room temperature with agitation (˜900 rpm on an orbital shaker).

After incubation with the detection reagent, the wells were washed three times as previously. The amount of bound label was then quantified by viewing the microparticles using a fluorescent microscope and determining the average levels of fluorescent signal binding to the microparticles of different codes.

In addition to the above, three combined variables were computed: (1) total IgA, (2) total IgG2 and (3) total IgM. These were calculated by summing the 16 variables for which (respectively) IgA, IgG2 and IgM immunoglobulin classes were detected.

Results: The following variables were found to be significantly different between the cancer samples and the control group samples, but not between the two control sample groups:
1. IgA vs. P1 antigen
2. IgA vs. Lewis-X antigen
3. IgA vs. Lewis-A antigen
4. IgA vs. Blood group H
5. IgA vs. Lewis-B antigen
6. IgA vs. Blood group A trisaccharide
7. IgA vs. Gal_{α1-2}Gal
8. IgG2 vs. Blood group A trisaccharide
9. IgG2 vs. Gal_{α1-3}Gal_{β1-}GlcNAc
10. IgG2 vs. α-Gal linear B trisaccharide
11. IgM vs. P1 antigen
12. IgM vs. Blood group A trisaccharide
13. IgM vs. Gal_{α1-2}Gal
14. IgM vs. Gal_{α1-3}Gal_{β1-3}GlcNAc
15. IgM vs. Gal_{α1-3}Gal
16. IgM vs. Gal_{α1-3}Gal_{β1-4}GlcNAc_{β1-3}Gal_{β1-4}Glc
17. Total IgA
18. Total IgG2
19. Total IgM.

In addition, potential causes of the differences seen were examined and it was concluded that the differences between patients and controls was not due to differences in age, sex, BMI, smoking, alcohol intake, year of sample collection, other concomitant diseases or medication between the sample groups.

The colon cancer pattern is also likely not related to location of the adenocarcinoma (rectum vs. colon), but there may be a marginally enhanced pattern in patients with more severe disease (stage III cancer vs. stage II).

Conclusions: Example 2 adds to the data of Example 1 in showing that the non-IgG immunoglobulins IgA and IgM binding to carbohydrate-containing epitopes is useful for the detection of colon cancer. Colon cancer and breast cancer are quite distinct in their pathogenesis and molecular physiology. In particular, markers currently used for one of them, such as CEA, are not useful for detecting the other. As a result, the demonstration that non-IgG immunoglobulins against carbohydrate-containing antigens are significantly lower in patients suffering either one of these distinct cancer types, provides strong evidence that low levels of non-IgG immunoglobulins against carbohydrate-containing antigens is associated with the risk of, or presence of, cancer per se, rather than with the specific location of the tumour, or the underlying pathophysiology of a particular tumour type.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

1. A method for identifying whether a mammal is suffering from, or at risk from, any form of cancer, wherein the method comprises:
(a) measuring a signal due to a non-IgG immunoglobulin binding to a carbohydrate-containing antigen in a sample from the mammal; and
(b) comparing the signal measured in (a) with a signal due to the non-IgG immunoglobulin binding to the carbohydrate-containing antigen in one or more samples from one or more mammals known to have cancer and/or with a signal due to the non-IgG immunoglobulin binding to the carbohydrate-containing antigen in one or more samples from one or more healthy mammals.

2. The method according to claim 1, wherein the measuring is performed in vitro.

3. The method of claim 1, wherein the signal due to non-IgG immunoglobulin binding to a carbohydrate containing antigen is measured in the following:
(i) binding the carbohydrate-containing antigen to a suitable substrate to form a coated substrate;
(ii) exposing the coated substrate to the sample; and
(iii) detecting non-IgG immunoglobulin bound to the coated substrate.

4. The method of claim 1, wherein the non-IgG immunoglobulin is one or more of the group consisting of IgA1, IgA2, total IgA, IgD, IgE and IgM.

5. The method of claim 1, wherein the non-IgG immunoglobulin is IgA.

6. The method of claim 1, wherein the carbohydrate-containing antigen is a tumour-associated cell surface antigen.

7. The method of claim 1, wherein the carbohydrate-containing antigen is α-gal, Lewis-A, Sialyl-Lewis-A, Lewis-X, Sialyl-Lewis-X, Tn, Sialyl-Tn or TF antigen.

8. The method of claim 7, wherein the carbohydrate-containing antigen is α-gal, TF antigen, or Sialyl-Lewis A.

9. The method of claim 8, wherein the cancer is breast cancer.

10. The method of claim 1, wherein the carbohydrate-containing antigen is P1 antigen, Lewis-X, Blood group H, Lewis-A, Lewis-B, Blood group A trisaccharide, or Gal_{α1-2}Gal.

11. (canceled)

12. The method of claim 1, wherein the carbohydrate-containing antigen is P1 antigen, Blood group A trisaccharide, Gal_{α1-2}Gal, Gal_{α1-3}Gal_{β1-3}GlcNAc, Gal_{α1-3}Gal, or Gal_{α1-3}Gal_{β1-4}GlcNAc_{β1-3}Gal_{β1-4}Glc.

13. (canceled)

14. The method of claim 12, wherein the non-IgG immunoglobulin is IgM.

15. The method of claim 10, wherein the cancer is colon cancer.

16. The method of claim 1, wherein the carbohydrate-containing antigen is coupled to a protein.

17. The method of claim 16, wherein the protein is serum albumin.

18. The method of claim 1, wherein more than one carbohydrate-containing antigen is used.

19. The method of claim 1, wherein more than one detection reagent specific for different classes of non-IgG immunoglobulin are used.

20. The method of claim 1, wherein the sample is serum, plasma or whole blood.

21. The method of claim 1, wherein the carbohydrate-containing antigen is α-gal and the non-IgG immunoglobulin is IgA.

22. The method of claim 1, wherein the carbohydrate-containing antigen is Lewis-A and the non-IgG immunoglobulin is IgA.

23. The method of claim 1, wherein the carbohydrate-containing antigen is P1 antigen and the non-IgG immunoglobulin is IgM.

24. The method of claim 1, wherein the cancer is selected from the group consisting of breast cancer, colon cancer, liver cancer, stomach cancer, ovarian cancer, brain cancer, pancreatic cancer, leukemia and bone cancer.

25. (canceled)

26. (canceled)

27. The method according to claim 1, wherein the mammal is a human.

28. A kit suitable for use in a method according to claim 1, the kit comprising:
(a) one or more carbohydrate-containing antigens; and
(b) one or more detection reagents capable of specifically recognizing one or more non-IgG immunoglobulins.

29. The kit according to claim 28, wherein the carbohydrate-containing antigens are selected from the group consisting of α-gal, Lewis-A, Sialyl-Lewis-A, Lewis-X, Sialyl-Lewis-X, Tn, Sialyl-Tn, TF antigen, P1 antigen, Blood group H, Lewis-B, Blood group A trisaccharide, Gal_{α1-2}Gal, Gal_{α1-3}Gal_{β1-3}GlcNAc, Gal_{α1-3}Gal, and Gal_{α1-3}Gal_{β1-4}GlcNAc_{β1-3}Gal_{β1-4}Glc.

30. The kit according to claim 28, wherein the detection reagent is specific for IgA1, IgA2, total IgA, IgD, IgE or IgM.

31. The kit according to 28, wherein the carbohydrate-containing antigens are selected from the group consisting of α-gal, Lewis-A, Sialyl-Lewis-A, Lewis-X, Sialyl-Lewis-X, Tn, Sialyl-Tn and TF antigen.

32. The kit according to claim 31, wherein the carbohydrate containing antigen is α-gal, the detection reagent is specific for total IgA (or IgA1 or IgA2), and the cancer is breast cancer.

33. The kit according to claim 28, wherein the carbohydrate-containing antigens are selected from the group consisting of P1 antigen, Lewis-X, Blood group H, Lewis-B, Blood group A trisaccharide and Gal_{α1-2}Gal.

34. The kit according to claim 33, wherein the carbohydrate containing antigen is Lewis-A, the detection reagent is specific for total IgA (or IgA1 or IgA2), and the cancer is colon cancer.

35. The kit according to claim 28, wherein the carbohydrate-containing antigens are selected from the group consisting of P1 antigen, Blood group A trisaccharide, Gal_{α1-2}Gal, Gal_{α1-3}Gal_{β1-3}GlcNAc, Gal_{α1-3}Gal, and Gal_{α1-3}Gal_{β1-4}GlcNAc_{β1-3}Gal_{β1-4}Glc.

36. The kit according to claim 35, wherein the carbohydrate containing antigen is P1 antigen, the detection reagent is specific for IgM, and the cancer is colon cancer.

37. The kit according to claim 28, wherein the detection reagent is an antibody.

38. The kit according to claim 28, wherein the detection reagent is labelled to facilitate quantitation.

39. The kit according to claim 28, having an additional component comprising an algorithm to classify a mammal (such as human) with respect to the presence of, or risk of, cancer based on the data obtained by applying the method of the invention to one or more samples from the mammal using the kit.
